(19) United States
(12) Patent Application Publication
(10) Pub. No.: US 2006/0134035 A1
(43) Pub. Date: Jun. 22, 2006

(54) Long wear topical composition having improved glossy appearance

(75) Inventors: Tao Zheng, Nanuet, NY (US); Giovana A. Sandstrom, Lodi, NJ (US)

Correspondence Address: Charles N.J. Ruggiero, Esq.;Ohlandt, Greeley, Ruggiero & Perle, L.L.P., 10th Floor, One Landmark Square, Stamford, CT, 06901-2682, US

(73) Assignee: Avon Products, Inc.

(21) Appl. No.: 11/019,708

(22) Filed: Dec. 22, 2004

(51) Int. Cl. A61K 8/89; A61K 8/81

(52) U.S. Cl. 424/64; 424/70.11; 424/70.12

(57) ABSTRACT

There are provided compositions and methods for imparting a long-wear and glossy effect to skin, lips, eyelashes, hair, and/or fingernails and toenails. The composition has hydrogenated polycyclopentadiene and a liquid gloss agent having a refractive index of at least 1.47. Preferably, the composition has phenylpropyldimethylsiloxysilicate, and is in a lipstick, a lip gloss, a mascara, an eye shadow, and/or a nail enamel composition.

### BACKGROUND OF THE INVENTION

1. Field of the Invention

The present invention relates to methods for cosmetically improving the appearance of skin, lips, eyelashes, hair, and/or fingernails and toenails. More particularly, the present invention relates to novel topical compositions that are useful to enhance the overall appearance of the skin, lips, eyelashes, hair, and/or fingernails and toenails, by imparting a long-wear and glossy finish.

2. Description of the Related Art

Many conventional cosmetics such as lipstick, lip gloss, mascara, eyeliner, and eye shadow, have a matte appearance upon application and drying. After application, the cosmetic quickly loses its fresh, glossy, or wet look and subsequently appears dull and dry regardless of the addition of pigments and other emollients. For example, conventional mascara formulations have a matte appearance upon application and drying on eyelashes. The wet or glossy look of freshly applied mascara quickly fades and as a result the eyes are left with a dull or dry appearance. This may be due, for example, to the combination of ingredients in a particular cosmetic composition formula.

Therefore, there remains a need to develop a glossy or wet look appearance for cosmetic compositions. Preferably, a glossy or wet look cosmetic composition would result in a fresh, healthy, and just-applied look, long after the cosmetic composition was applied and had dried. The need for improved gloss in cosmetic compositions has resulted in many gloss agents, such as dimethicone and fluorosilicones, in the cosmetic raw ingredient market.

Another desirable attribute for cosmetic formulations is to provide a long-wear and water-resistant effect to cosmetics that are applied to the skin, lips, eyelashes, hair, and/or fingernails and toenails. It is known in the art that hydrogenated polycyclopentadiene is a long-wear film former that improves the adhesion and substantivity of the cosmetic film to the skin, lips, eyelashes, hair, or nails. It also improves the water-resistance of the film. However, cosmetic films containing hydrogenated polycyclopentadiene alone do not provide sufficient shining appearance to fulfill the high gloss trend in many cosmetic markets. Furthermore, mixing conventional gloss agents with hydrogenated polycyclopentadiene results in phase separation, and loss of both long-wear and gloss effects.

Clearly, there are competing factors in the development of cosmetic compositions that simultaneously provide for long-wear and high gloss that, heretofore, have been extremely difficult, if not impossible, to resolve.

The present invention is a composition for a cosmetic for application to skin surfaces, such as the face and lips. The composition can also be used in formulations for application to the eyelashes, hair, and/or fingernails and toenails.

It would be desirable to have cosmetic compositions that provide both long-wear and high gloss effects to skin, lips, eyelashes, hair, and/or fingernails and toenails, from a single composition. It would be further desirable to have cosmetic compositions that combine long-wear film formers with compatible gloss agents.

### SUMMARY OF THE INVENTION

It is an object of the present invention to provide compositions for imparting long-wear and glossy effects to skin, lips, eyelashes, hair, and/or fingernails and toenails.

It is another object of the present invention to provide compositions for imparting long-wear and glossy effects, that have both long-wear film formers and gloss agents that are compatible with one another in a single composition.

It is still another object of the present invention to provide compositions for imparting long-wear and glossy effects, that can be present in a homogenous clear mixture.

According to this and other objects and advantages of the present invention, there are provided compositions for imparting a long-wear and glossy effect to skin, lips, eyelashes, hair, and/or fingernails and toenails, and methods for applying same. The compositions have hydrogenated polycyclopentadiene and a liquid gloss agent having a refractive index of at least 1.47. A preferred composition is a lip gloss composition that has phenylpropyldimethylsiloxysilicate as the gloss agent.

Further according to this and other objects and advantages of the present invention, there are also provided methods for imparting a long-wear and glossy effect to skin, lips, eyelashes, hair, and/or fingernails and toenails. A method includes applying to the skin, lips, eyelashes, hair, and/or fingernails and toenails, a composition having hydrogenated polycyclopentadiene and a liquid gloss agent having a refractive index of at least 1.47.

### DETAILED DESCRIPTION OF THE INVENTION

The present invention provides compositions that impart long-wear and glossy effects to skin, lips, eyelashes, hair, and/or fingernails and toenails. The term “nails” is used throughout to refer to fingernails and/or toenails. A composition of the present invention can be a lip color or gloss product, such as a lipstick or lip gloss. The composition can also be incorporated in hair styling products, such as gels, leave-in conditioners, hair sprays, or temporary hair colorants. Also, the composition can be incorporated in skin coloring products such as eye shadows. Additionally, the composition can be incorporated in fingernail and toenail polish products, such as nail enamel. Furthermore, the composition can also be incorporated into eyelash products, such as mascaras. The present invention also includes methods for imparting long-wear and glossy effects to skin, lips, eyelashes, hair, and/or nails by applying one of the compositions thereto.

Preferably, the compositions have hydrogenated polycyclopentadiene in an amount about 0.1 percent by weight (wt %) to about 50 wt % based on the total weight of the composition. More preferably, the compositions have hydrogenated polycyclopentadiene in an amount about 0.5 wt % to about 25 wt % based on the total weight of the composition. Most preferably, the compositions have hydrogenated polycyclopentadiene in an amount about 1 wt % to about 15 wt % based on the total weight of the composition.

The compositions also have a liquid gloss agent with a refractive index of at least 1.47, preferably in an amount about 1 wt % to about 50 wt % based on the total weight of the composition. More preferably, the compositions have a liquid gloss agent in an amount about 5 wt % to about 30 wt % based on the total weight of the composition. Most preferably, the compositions have a liquid gloss agent in an amount about 10 wt % to about 20 wt % based on the total weight of the composition. The liquid gloss agent is compatible with hydrogenated polycyclopentadiene. Gloss agents that can be used in the present compositions include, but are not limited to, phenylpropyldimethylsiloxysilicate, polybutene, polyisobutene, hydrogenated polyisobutene, or any combinations thereof. Polybutene is a polymer formed by the polymerization of a mixture of iso- and normal butenes.

Hydrogenated polycyclopentadiene (e.g. KOBOGUARD 5400, distributed by Kobo Products, Inc.) is known to those of ordinary skill in art of cosmetics as a long-wear film former in cosmetic formulations. It is available as 70% solution in isododecane (70% solid). KOBOGUARD 5400 is also available as 50% solution in squalene (50% solid), and 50% solution in isooctane (50% solid). KOBOGUARD formula (isododecane, C10-13 isoparaffin, isooctane) vehicles include various esters and oils, including caprylic/capric triglyceride, neopentyl glycol diheptanoate, octyl palmitate, squalane and mineral oil.

Hydrogenated polycyclopentadiene improves the adhesion and substantivity of the cosmetic film to the skin, lips, eyelashes, hair, or nails. It also improves the water-resistance of the film. However, cosmetic film containing hydrogenated polycyclopentadiene alone does not provide enough shining appearance to fulfill the high gloss trend in many cosmetic markets.

There are also many gloss agents available in the raw ingredient market. Some of these gloss agents, for example, dimethicone and fluorosilicone, are not compatible with hydrogenated polycyclopentadiene. Mixing these gloss agents with hydrogenated polycyclopentadiene results in phase separation, and loss of both long-wear and gloss effects.

The present compositions provide for long-wear and high glossy effects from a single composition. What the present invention has discovered is that some special gloss agents, for example, phenylpropyldimethylsiloxysilicate (e.g. SILSHINE 151), polybutene, and hydrogenated polyisobutene, are compatible with hydrogenated polycyclopentadiene. The compatible system is preferably a homogenous clear mixture. A simple formula having this combination results in a long-wear and high gloss film. A synergistic effect can be created between hydrogenated polycyclopentadiene and a compatible gloss agent with a refractive index of at least 1.47. Preferably, the present compositions have phenylpropyldimethylsiloxysilicate, which has a refractive index of about 1.518, higher than that of most gloss agents. Polybutene and hydrogenated polyisobutene, which may also be used, have a refractive index of about 1.498 and about 1.495, respectively. Preferably, gloss agents having a refractive index from about 1.47 to about 1.65 may be used.

Phenylpropyldimethylsiloxysilicate is compatible with many silicones and organics. It can also function as a co-solubilizer for hydrogenated polycyclopentadiene with other chemicals. Phenylpropyldimethylsiloxysilicate is a phenyl substituted MQ resin. However, it was discovered that the regular MQ resin, trimethylsiloxysilicate, is not compatible with hydrogenated polycyclopentadiene.

The present compositions typically have a vehicle or carrier. The vehicle should be a physiologically acceptable or suitable vehicle. A “physiologically acceptable vehicle” or a “suitable vehicle” means any vehicle for a drug, cosmetic or medicament that is suitable for use in direct, safe contact with human tissues. The present composition is preferably incorporated into a suitable topical vehicle to form a topical formulation prior to applying.

In the present compositions, the vehicle may be in the form of an aqueous phase, an oil phase, a gel, a wax-in-water emulsion, an oil-in-water emulsion, or a water-in-oil emulsion. The aqueous phase is a mixture of one or more water soluble or water dispersible ingredients, which can be liquid, semi-solid or solid at room temperature (25° C.). The vehicle comprises or can be in the form of a suspension, dispersion or solution in water or an aqueous-alcoholic vehicle, which may contain a thickener or gellant. A person skilled in the art can select the appropriate cosmetic form, the ingredients contained therein, as well as the method for preparing it, on the basis of the knowledge that the skilled artisan possesses.

The compositions may include an aqueous phase which may contain water or a mixture of water and at least one hydrophilic organic solvent, in particular an alcohol especially a linear or branched lower monoalcohol containing from 2 to 5 carbon atoms, e.g., ethanol or propanol; a polyol, e.g., propylene glycol, sorbitol, glycerol, diglycerol, panthenol, or polyethylene glycol, or any mixtures therof. This aqueous phase may represent from about 0.5 wt % to about 99.99 wt % based on the total weight of the composition.

When the present compositions are in the form of an emulsion, such composition can also optionally comprise a surfactant, preferably in an amount from about 0.1 wt % to about 30 wt %, and more preferably from about 1 wt % to about 20 wt %, based on the total weight of the composition.

The compositions can also comprise a film-forming polymer such as a polyurethane, a polyacrylic homopolymer or copolymer, a polyester, nitrocellulose, a hydrocarbon-based resin and/or a silicone resin. This is especially the situation when it is desired to prepare a composition such as a lip stick, a water-based nail varnish, or a mascara. The polymers can be dissolved or dispersed in the cosmetically acceptable vehicle and optionally combined with a plasticizer.

Present compositions may also comprise an oil phase containing oil soluble or oil dispersible ingredients that are liquid at room temperature (25° C.) and/or oily or waxy substances that are solid at room temperature, such as waxes, semisoilids, gums, and mixtures thereof. This oily phase may also contain organic solvents.

Suitable oily materials that are liquid at room temperature, often referred to as oils, include hydrocarbon-based oils of animal origin such as perhydrosqualene; hydrocarbon-based plant oils such as liquid triglycerides of fatty acids of 4 to 10 carbon atoms, for instance, heptanoic or octanoic acid triglycerides; caprylic/capric acid triglycerides; or oils such as sunflower oil, corn oil, soybean oil, grapeseed oil, castor oil, avocado oil, jojoba oil; linear or branched hydrocarbons of mineral or synthetic origin such as liquid paraffins and derivatives thereof; petroleum jelly; synthetic esters and ethers, in particular esters of fatty, namely, for example, isopropyl myristate, 2-ethylhexyl palmitate, 2-octyldodecyl stearate, isostearyl isostearate; hydroxylated esters such as isostearyl lactate, octyl hydroxystearate, octyldodecyl hydroxystearate, heptanoates, octanoates and decanoates of fatty alcohols; polyol esters such as propylene glycol dioctanoate, neopentyl glycol diheptanoate, diethylene glycol diisononanoate; and pentaerythritol esters; fatty alcohols containing from 12 to 26 carbon atoms such as octyldodecanol, 2-butyloctanol, 2-hexyldecanol, 2-undecylpentadecanol, oleyl alcohol; partially hydrocarbon-based fluoro oils and/or fluorosilicone oils; silicone oils such as volatile or non-volatile, linear or cyclic polymethylsiloxanes (PDMS) that are liquid or semisolid at room temperature such as cyclomethicones and dimethicones, optionally comprising a phenyl group, for instance phenyl trimethicones, siloxanes, and mixtures thereof. These oils are usually present in an amount from about 0 wt % to about 90 wt %, and preferably from about 1 wt % to about 80 wt %, based on the total weight of the oil phase.

The oil phase of the present compositions may also comprise one or more cosmetically acceptable organic solvents. Preferably, these solvents are present at about 0 wt % to about 60 wt % based on the total weight of the composition. More preferably, the solvents are present at about 1 wt % to about 30 wt % based on the total weight of the composition. The solvents that can be used include, but are not limited to, lipophilic organic solvents, amphiphilic organic solvents, or mixtures thereof. Other suitable solvents which can be used in the compositions include, but are not limited to, acetic acid esters such as methyl, ethyl, butyl, amyl or 2-methoxyethyl acetate; ketones such as methyl ethyl ketone, methyl isobutyl ketone, isopropyl acetate; hydrocarbons such as toluene, xylene, p-cylene, hexane or heptane; aldehydes containing from 5 to 10 carbon atoms; ethers containing at least 3 carbon atoms; or mixtures thereof.

The present compositions may further comprise any ingredient conventionally used in the cosmetic field. These ingredients include preserving agents, aqueous phase thickeners (polysaccharide biopolymers, synthetic polymers) and fatty-phase thickeners, fragrances, hydrophilic and lipophilic active agents, or mixtures thereof. The amounts of these various ingredients are those conventionally used in the cosmetic field to achieve their intended purpose, and range typically from about 0.01 wt % to about 20 wt % based on the total weight of the composition. The nature of these ingredients and their amounts must be compatible with the production of stable, thickened, glossy compositions of the invention.

The compositions may also comprise an additional particulate phase, typically present at about 0 wt % to about 30 wt % based on the total weight of the composition. Preferably, the particulate phase is present at about 0.05 wt % to about 20 wt % based on the total weight of the composition, and can comprise pigments and/or pearlescent agents and/or fillers used in cosmetic compositions.

Colorants are any conventional colorant in any cosmetic composition that is used for the purpose of exhibiting color on skin, hair, or lips. Colorants that can be used in the present compositions include, but are not limited to, D&C Red No. 3, D&C Red No. 6, D&C Red No. 7, D&C Red No. 8, D&C Red No. 9, D&C Red No. 21, D&C Red 22, D&C Red No. 27, D&C Red 28, D&C Red No. 30, D&C Red No. 33, D&C Red 34, D&C Red No. 36, FD&C Red No. 40, D&C Yellow No. 5, FD&C Yellow No. 5, FD&C Yellow No. 6, D&C Yellow No. 10, D&C Green No. 3, D&C Green No. 5, D&C Orange No. 5, FD&C Blue No. 1, annatto, copper powder, bismuth oxychloride, guanine, bronze powder, iron oxides, carmel, manganese violet, carmine, mica, titanium dioxide-coated mica, carotene, titanium dioxide, chlorophyllin-calcium complex, zinc oxide, or any combinations thereof. Also suitable are chemically treated pigments such as isopropyl titanium triisostearate (ITT) (e.g. titanate from Kobo Products, Inc.), magnesium myristate, triethoxy caprylylsilane (e.g. silane from Kobo Products, Inc.), silicone (including methicone and dimethicone), C9-15 fluoroalcohol phosphates (e.g. perfluoroalcohol phosphate from Kobo Products, Inc.), acrylates/dimethicone copolymer (e.g. acrylic silicone copolymer (ASC) from Kobo Products, Inc.), C9-15 fluoroalcohol phosphates (methicone and alumina) (e.g. PF—Si hybrid (perfluoroalcohol phosphate-silicone treatment) from Kobo Products, Inc.), C9-15 fluoroalcohol phosphates (acrylates/dimethicone copolymer) (e.g. PF-ASC hybrid (perfluoroalcohol phosphate-acryl silicone copolymer) from Kobo Products, Inc.), lecithin, carnauba wax, polyethylene, chitosan, and lauroyl-lysine. Such chemically treated pigments are known to those of ordinary skill in the art and/or are listed in Kobo Products, Inc.'s Treated Pigments brochure (May 2000).

The term “pigments” should be understood as meaning white or colored, inorganic or organic particles that are insoluble in the liquid hydrophilic phase and which are intended to color and/or opacity the composition. The term “fillers” should be understood as meaning colorless or white, inorganic or synthetic, lamellar or non-lamellar particles. The term “pearlescent agents” should be understood as meaning iridescent particles, in particular produced by certain molluscs in their shell or synthesized pearlescent particles. These fillers and pearlescent agents serve in particular to modify the texture of the composition.

Pigments can be present in the composition in an amount at about 0 wt % to about 25 wt %, and preferably in an amount at about 2 wt % to about 15 wt %, based on the total weight of the final composition. Suitable inorganic pigments include titanium oxide, zirconium oxide and cerium oxide, as well as zinc oxide, iron oxide, chromium oxide and ferric blue. Suitable organic pigments which can be used include carbon black and barium, strontium, calcium or aluminium lakes or alternatively the diketopyrrolopyrroles (DPP) described in EP 0 542 669, EP 0 787 730, EP 0 787 731 and WO 96/08537.

Pearlescent agents can be present in the composition at about 0 wt % to about 20 wt % based on the total weight of the composition. Preferably, the pearlescent agents are present at about 1 wt % to about 15 wt % based on the total weight of the composition. Suitable pearlescent agents include mica coated with titanium oxide, with iron oxide, with natural pigment or with bismuth oxychloride, such as colored titanium mica.

Fillers are normally present at about 0 wt % to about 30 wt % based on the total weight of the composition. Preferably the fillers are present at about 0.5 wt % to about 15 wt % based on the total weight of the composition. Suitable fillers include, but are not limited to, talc, silica, zinc stearate, mica, kaolin, nylon (in particular ORGASOL) powder, polyethylene powder, TEFLON, starch, boron nitride, copolymer microspheres such as EXPANCEL (Nobel Industries), POLYTRAP (Dow Corning), and silicone resin microbeads (TOSPEARL from Toshiba).

The oil phase of the present compositions may comprise one or more waxes, gums, or mixtures thereof. The waxes include hydrocarbon-based waxes, fluoro waxes and/or silicone waxes and can be of plant, mineral, animal and/or synthetic origin. In particular, the waxes have a melting point of greater than 25° C., preferably greater than 45° C. Suitable waxes include beeswax, carnauba wax, candelilla wax, paraffin wax, microcrystalline wax, ceresin, ozokerite wax; synthetic wax such as polyethylene wax, and silicone wax containing from 16 to 45 carbon atoms.

The gums are generally high molecular weight PDMSs or cellulose gums or polysaccharides and the semisolid materials are generally hydrocarbon-based compounds such as lanolins and derivatives thereof or alternatively PDMSs.

The compositions of the present invention typically have from about 0 to about 40 wt % waxes based on the total weight of the composition. Preferably, the waxes are present from about 1 wt % to about 25 wt % based on the total weight of the composition.

The compositions may have other ingredients such as one or more anesthetics, anti-allergenics, antifungals, anti-inflammatories, antimicrobials, antiseptics, chelating agents, emollients, emulsifiers, fragrances, humectants, lubricants, masking agents, medicaments, moisturizers, pH adjusters, preservatives, protectants, soothing agents, stabilizers, sunscreens, surfactants, thickeners, viscosity control agents, vitamins, pigments, bioactives such as those providing lip plumping or anti-wrinkle effect, or any combinations thereof. Such ingredients are known to those of ordinary skill in the art and/or are listed in the INCI Dictionary and Handbook, 10^{th} Edition, Volume 3 (2004).

The compositions can take any semisolid or liquid product form suitable for application to the skin, lips, eyelashes, hair, and/or nails, such as a cream, ointment, solution, paste, pomade, gel, lotion, or mascara. Preferred compositions are long-wear and glossy lip-coloring lipsticks, long-wear and glossy lip glosses, long-wear and glossy mascaras, long-wear and glossy eye shadows, and long-wear and glossy nail enamels. The compositions can have the appearance of a cream, salve, fluid lotion, soft paste, ointment or a solid which is poured or cast and in particular as a stick or a dish.

Gloss is an optical phenomenon that can be evaluated by examining the appearance of a surface. The evaluation of gloss describes the capacity of a surface to reflect directed light. The appearance of gloss is influenced by several factors including: material (e.g. glass, coatings, metals), surface profile (e.g. flat, structured), transparency and substrate, type of illumination, and the observer.

In the case of high gloss surfaces, gloss is determined by direct reflection on a first surface. Light reflected from the surface follows the reflection law (angle of illumination equals the angle of reflection). The intensity of the reflected light is dependent on the angle of illumination and material properties.

In the case of metal and coating surfaces, color results in diffuse reflection from within the sample. Part of the illumination penetrates the surface and is selectively absorbed and scattered internally by pigment particles and other bodies. It is then diffused from within the first surface. This is how the impression color is caused.

In the case of glossy surfaces with haze, the dominant part of light is reflected in the main direction (specular). A small amount of light is scattered in directions adjacent to the direction of specular reflection. This scattered light of low intensity causes haze. The surface appears to be glossy, yet has a milky appearance.

In the case of medium to mat surfaces, light is not only reflected in the direction of specular reflection, but also in other directions. The capacity of a surface to reflect a light source or other images is strongly reduced. The more evenly the intensity is distributed in all directions, the less glossy a surface will appear.

In measuring gloss, the angle of illumination highly influences the measurement results. In order to evaluate the whole range from high-gloss to mat surfaces, three different angles of illumination (i.e. three different measuring ranges) are defined for the paint and coatings industry, as shown in Table 1.

TABLE 1
Gloss Measurement

| Gloss Level | 60° value | Recommended Geometry |
|---|---|---|
| Low gloss | <10 units | 85° geometry |
| Semi gloss | 10-70 units | 60° geometry |
| High gloss | >70 units | 20° geometry |

When preparing a sample, in order to achieve highly accurate and repeatable results, the test specimen material should be: flat, free of structures, similar in color and lightness, and non-luminescent. If the sample is pigmented, a clean glass plate should be used as a substrate. Wet film having 3 mil. thickness should be drawn down by an applicator. The sample is ready for gloss measurement after 24 hours drying process in room temperature. If the sample is non-pigmented, a Leneta card should be used as a substrate. Wet film with 3 mil. thickness should be drawn down by the applicator. The sample is ready for gloss measurement after 24 hours drying process in room temperature.

Gloss is measured by a Glossmeter (BYK-Gardner, MICRO-TRI-GLOSS, Model 4520). The device is calibrated before measurement. The gloss measurement is taken on the black side of the Lenata card, if the Lenata card is used as a substrate. In order to differentiate the gloss of samples, it is necessary to select the appropriate measuring geometry. First, the test specimen is measured with the 60° geometry. The 60° geometry is used if the gloss reading is between 10 and 70 units. If the 60° gloss is higher than 70 units, the 20° geometry is advantageous for comparison. If the 60° gloss is lower than 10 units, the 85° geometry is used. At least three readings are taken on a test specimen. If the range is greater than five gloss units, additional readings are taken. For correct measurement results, the mean gloss reading is calculated, and the geometry used is reported (e.g. 20° reflectometer value R′20=55 units).

Table 2 shows the gloss measurement results for examples of present compositions versus a control at 20° geometry. The control composition is KOBOGUARD 5400 IDD, which is 70% hydrogenated polycyclopentadiene dissolved in isododecane. The gloss measurement was taken on the black side of the Leneta card. Present compositions provide a 20° geometry gloss value increase of at least 30%, and a corresponding 20° geometry gloss value of at least 68.

TABLE 2
Gloss Measurements

| | Gloss | Gloss |
|---|---|---|
| | Value | Value |
| | In 20° | Increase |
| Composition | Geometry | (%) |
| KOBOGUARD 5400 IDD + Isododecane (1:1)¹ | 52.2 | Control |
| KOBOGUARD 5400 IDD + SILSHINE 151 (1:1) | 79.8 | 52.9% |
| KOBOGUARD 5400 IDD + Polybutene (1:1) | 76.2 | 46.0% |
| KOBOGUARD 5400 IDD + Hydrogenated | 75.4 | 44.4% |
| Polyisobutene (1:1) | | |

¹The ratio of hydrogenated polycyclopentadiene to isododecane is 0.7:1.3, after including the isododecane contribution from the KOBOGUARD 5400 IDD

The following are examples of compositions of the present invention and are not to be construed as limiting. Unless otherwise indicated, all percentages and parts are by weight. All ingredients are “as is” unless otherwise noted.

The following formulations were prepared. The ingredients were as set forth in Examples 1 and 2 below:

### EXAMPLE 1

Lip Gloss Simple Formulation

| Ingredient | Concentration (wt %) |
|---|---|
| Hydrogenated polycyclopentadiene | 25% |
| Isododecane | 35% |
| Phenylpropyldimethylsiloxysilicate | 25% |
| Polymethylsilsequioxane | 10% |
| Pigments | 5% |
| | 100% |

### EXAMPLE 2

Long Lasting Lip Gloss

| Ingredient | Concentration (wt %) |
|---|---|
| Polybutene¹ | qs 100% |
| Hydrogenated Polyisobutene² | 1.64% |
| Phenylpropyldimethylsiloxysilicate³ | 1.44% |
| Sucrose Acetete Isobutyrate In | 3.23% |
| Caprylic/Caprylate Triglycerides⁴ | |
| Butyl Acrylate/Hydroxypropyldimethicone | 1.4% |
| Acrylate Copolymer⁵ | |
| Hydrogrogenated Polycyclopentadiene/ | 15% |
| Isododecane Blend⁶ | |
| Acrylates Copolymer/Isododecane⁷ | 4.39% |
| Iron Oxides | 3.14% |
| D&C Red No. 7 | 1.34% |
| FD&C Blue No. 1 | 0.53% |
| Mica | 7.84% |
| Bismuth Oxychloride-Octagonal | 1.65% |
| Dimethicone/Divinyldimethicone/Silsesquioxane | 4% |
| Crosspolymer⁸ | |
| Lauryl Dimethicone/Vinyl Dimethicone | 11% |
| Crosspolymer/Squalane Blend⁹ | |
| Dimethicone Copolyol-Water Dispersible¹⁰ | 10% |
| Montmorillonite/PEG-30 Dipolyhydroxy Stearate/ | 3.8% |
| Ethylhexyl. Stearate/Propylene Carbonate Blend¹¹ | |
| Isododecane | 4% |
| | 100% |

¹Exxon
²Uniquema
³Kobo
⁴Eastman
⁵Grant
⁶Kobo
⁷Phoenix
⁸Grant
⁹ShinEtsu
¹⁰Dow Corning
¹¹Amcol

### EXAMPLE 3

Nail Enamel

| Ingredient | Concentration (wt %) |
|---|---|
| Ethyl Acetate | 38% |
| Polyurethane-8 (27.5%)/EthylAcetate/Isopropyl | 30% |
| Alcohol¹ | |
| KOBOGUARD Isoctane² | 14% |
| Polybutene³ | 5% |
| Tosylamide/Epoxy Resin (Aromatic)/Butyl | 4% |
| Acetate⁴ | |
| Acetyl Tributyl Citrate | 4% |
| Butyl Acetate | 3% |
| Sucrose Benzoate | 2% |
| | 100% |

¹Kane International
²Kobo
³Exxon
⁴Estron Chemical

The composition of Example 2 is applied to lips. Upon application of the composition, the hydrogenated polycyclopentadiene and phenylpropyldimethylsiloxysilicate forms a long-wearing glossy film on the lips.

It should be understood that the foregoing description is only illustrative of the present invention. Various alternatives and modifications can be devised by those skilled in the art without departing from the invention. Accordingly, the present invention is intended to embrace all such alternatives, modifications and variances that fall within the scope of the appended claims.

What is claimed is:

1. A cosmetic composition with a long-wear and glossy effect comprising:
hydrogenated polycyclopentadiene; and
a liquid gloss agent having a refractive index of at least 1.47.

2. The composition of claim 1, wherein the liquid gloss agent is compatible with hydrogenated polycyclopentadiene.

3. The composition of claim 1, wherein the liquid gloss agent is selected from the group consisting of phenylpropyldimethylsiloxysilicate, polybutene, polyisobutene, hydrogenated polyisobutene, and any combinations thereof.

4. The composition of claim 1, wherein the liquid gloss agent is phenylpropyldimethylsiloxysilicate.

5. The composition of claim 1, wherein the liquid gloss agent has a refractive index of about 1.47 to about 1.65.

6. The composition of claim 1, wherein the liquid gloss agent has a refractive index of 1.518.

7. The composition of claim 1, wherein the liquid gloss agent is present at about 1 wt % to about 50 wt % based on the total weight of the composition.

8. The composition of claim 1, wherein the liquid gloss agent is present at about 5 wt % to about 30 wt % based on the total weight of the composition.

9. The composition of claim 1, wherein the liquid gloss agent is present at about 10 wt % to about 20 wt % based on the total weight of the composition.

10. The composition of claim 1, wherein the hydrogenated polycyclopentadiene is present at about 0.1 wt % to about 50 wt % based on the total weight of the composition.

11. The composition of claim 1, wherein the hydrogenated polycyclopentadiene is present at about 0.5 wt % to about 25 wt % based on the total weight of the composition.

12. The composition of claim 1, wherein the hydrogenated polycyclopentadiene is present at about 1 wt % to about 15 wt % based on the total weight of the composition.

13. The composition of claim 1, wherein the composition is a homogenous clear mixture.

14. The composition of claim 1, further comprising a vehicle selected from the group consisting of aqueous phase, oil phase, gel, wax-in-water emulsion, oil-in-water emulsion, water-in-oil emulsion, suspension, dispersion, solution in water, aqueous-alcoholic vehicle, and any combinations thereof.

15. The composition of claim 1, further comprising a colorant selected from the group consisting of D&C Red No. 3, D&C Red No. 6, D&C Red No. 7, D&C Red No. 8, D&C Red No. 9, D&C Red No. 21, D&C Red 22, D&C Red No. 27, D&C Red 28, D&C Red No. 30, D&C Red No. 33, D&C Red 34, D&C Red No. 36, FD&C Red No. 40, D&C Yellow No. 5, FD&C Yellow No. 5, FD&C Yellow No. 6, D&C Yellow No. 10, D&C Green No. 3, D&C Green No. 5, D&C Orange No. 5, FD&C Blue No. 1, annatto, copper powder, bismuth oxychloride, guanine, bronze powder, iron oxide, carmel, manganese violet, carmine, mica, titanium dioxide-coated mica, carotene, titanium dioxide, chlorophyllin-calcium complex, zinc oxide, and any combinations thereof.

16. The composition of claim 1, further comprising an inorganic pigment selected from the group consisting of titanium oxide, zirconium oxide cerium oxide, zinc oxide, iron oxide, chromium oxide, ferric blue, and any combinations thereof.

17. The composition of claim 1, further comprising an organic pigment selected from the group consisting of carbon black, barium, strontium, calcium lake, aluminium lake, diketopyrrolopyrrole, and any combinations thereof.

18. The composition of claim 1, further comprising one or more additional components selected from the group consisting of anesthetics, anti-allergenics, antifungals, anti-inflammatories, antimicrobials, antiseptics, chelating agents, emollients, emulsifiers, fragrances, humectants, lubricants, masking agents, medicaments, moisturizers, pH adjusters, preservatives, protectants, soothing agents, stabilizers, sunscreens, surfactants, thickeners, viscosity control agents, vitamins, pigments, bioactives, and any combinations thereof.

19. The composition of claim 1, wherein the composition is selected from the group consisting of a lip gloss composition, a lip coloring composition, a mascara, an eye shadow, and a nail enamel.

20. A method for imparting a long-wear and gloss effect to skin, lips, eyelashes, hair, or nails, comprising topically applying a cosmetic composition having:
hydrogenated polycyclopentadiene; and
a liquid gloss agent having a refractive index of at least 1.47.

21. The method of claim 20, wherein the liquid gloss agent is selected from the group consisting of phenylpropyldimethylsiloxysilicate, polybutene, polyisobutene, hydrogenated polyisobutene, and any combinations thereof.

22. The method of claim 20, wherein the liquid gloss agent is phenylpropyldimethylsiloxysilicate.

23. The method of claim 20, wherein the liquid gloss agent has a refractive index of about 1.47 to about 1.65.

24. The method of claim 20, wherein the liquid gloss agent has a refractive index of 1.518.

25. The method of claim 20, wherein the liquid gloss agent is present at about 1 wt % to about 50 wt % based on the total weight of the composition.

26. The method of claim 20, wherein the liquid gloss agent is present at about 5 wt % to about 30 wt % based on the total weight of the composition.

27. The method of claim 20, wherein the liquid gloss agent is present at about 10 wt % to about 20 wt % based on the total weight of the composition.

28. The method of claim 20, wherein the hydrogenated polycyclopentadiene is present at about 0.1 wt % to about 50 wt % based on the total weight of the composition.

29. The method of claim 20, wherein the hydrogenated polycyclopentadiene is present at about 0.5 wt % to about 25 wt % based on the total weight of the composition.

30. The method of claim 20, wherein the hydrogenated polycyclopentadiene is present at about 1 wt % to about 15 wt % based on the total weight of the composition.

31. The method of claim 20, wherein the composition is a homogenous clear mixture.

32. The method of claim 20, wherein the composition further comprises a vehicle selected from the group consisting of aqueous phase, oil phase, gel, wax-in-water emulsion, oil-in-water emulsion, water-in-oil emulsion, suspension, dispersion, solution in water, aqueous-alcoholic vehicle, and any combinations thereof.

33. The method of claim 20, wherein the composition further comprises a colorant selected from the group consisting of D&C Red No. 3, D&C Red No. 6, D&C Red No. 7, D&C Red No. 8, D&C Red No. 9, D&C Red No. 21, D&C Red 22, D&C Red No. 27, D&C Red 28, D&C Red No. 30, D&C Red No. 33, D&C Red 34, D&C Red No. 36, FD&C Red No. 40, D&C Yellow No. 5, FD&C Yellow No. 5, FD&C Yellow No. 6, D&C Yellow No. 10, D&C Green No. 3, D&C Green No. 5, D&C Orange No. 5, FD&C Blue No. 1, annatto, copper powder, bismuth oxychloride, guanine, bronze powder, iron oxide, carmel, manganese violet, carmine, mica, titanium dioxide-coated mica, carotene, titanium dioxide, chlorophyllin-calcium complex, zinc oxide, and any combinations thereof.

34. The method of claim 20, wherein the composition further comprises one or more additional components selected from the group consisting of anesthetics, anti-allergenics, antifungals, anti-inflammatories, antimicrobials, antiseptics, chelating agents, emollients, emulsifiers, fragrances, humectants, lubricants, masking agents, medicaments, moisturizers, pH adjusters, preservatives, protectants, soothing agents, stabilizers, sunscreens, surfactants, thickeners, viscosity control agents, vitamins, pigments, bioactives, and any combinations thereof.

35. The method of claim 20, wherein the composition is applied to the skin.

36. The method of claim 20, wherein the composition is applied to the lips.

37. The method of claim 20, wherein the composition is applied to the eyelashes.

38. The method of claim 20, wherein the composition is applied to the hair.

39. The method of claim 20, wherein the composition is applied to the nails.

40. The method of claim 20, wherein the composition is selected from the group consisting of a lip gloss composition, a lip coloring composition, a mascara, an eye shadow, and a nail enamel.
